(19) United States
(12) Patent Application Publication
(10) Pub. No.: US 2009/0093495 A1
(43) Pub. Date: Apr. 9, 2009

(54) COMBINATION OF A SRC KINASE INHIBITOR AND A BCR-ABL INHIBITOR FOR THE TREATMENT OF PROLIFERATIVE DISEASES

(75) Inventor: Francis Y. Lee, Yardley, PA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 12/254,896

(22) Filed: Oct. 21, 2008

Related U.S. Application Data
(63) Continuation of application No. 11/265,843, filed on Nov. 3, 2005, now abandoned.
(60) Provisional application No. 60/624,937, filed on Nov. 4, 2004.
(60) Provisional application No. 60/632,122, filed on Dec. 1, 2004.
(60) Provisional application No. 60/649,722, filed on Feb. 3, 2005.
(60) Provisional application No. 60/703,628, filed on Jul. 29, 2005.

(51) Int. Cl. A61K 31/496; A61P 35/00

(52) U.S. Cl. 514/252.18

(57) ABSTRACT

A combination and methods are disclosed which are useful for the treatment of cancer and/or leukemia.

### CROSS-REFERENCE TO RELATED APPLICATIONS

This application is a continuation of U.S. application Ser. No. 11/265,843, filed Nov. 3, 2005, which claims the priority benefit of U.S. Provisional Application Ser. No. 60/624,937 filed Nov. 4, 2004, U.S. Provisional Application Ser. No. 60/632,122 filed Dec. 1, 2004, U.S. Provisional Application Ser. No. 60/649,722 filed Feb. 3, 2005, and U.S. Provisional Application Ser. No. 60/703,628 filed Jul. 29, 2005, the entire disclosures of which are herein incorporated by reference in their entirety.

### FIELD OF THE INVENTION

This invention relates to the fields of oncology and improved chemotherapy regimens.

### BACKGROUND OF THE INVENTION

The disclosure of each literature article and published patent document referred to herein is incorporated by reference herein in its entirety.

The National Cancer Institute has estimated that in the United States alone, 1 in 3 people will be struck with cancer during their lifetime. Moreover, approximately 50% to 60% of people contracting cancer will eventually succumb to the disease. The widespread occurrence of this disease underscores the need for improved anticancer regimens for the treatment of malignancy.

Due to the wide variety of cancers presently observed, numerous anticancer agents have been developed to destroy cancer within the body. These compounds are administered to cancer patients with the objective of destroying or otherwise inhibiting the growth of malignant cells while leaving normal, healthy cells undisturbed. Anticancer agents have been classified based upon their mechanism of action.

The present invention is directed to Src Kinase Inhibitors in combination with a BCR-ABL kinase inhibitor.

With the near completion of the Human Genome Project, it can be estimated that the human genome encodes close to 100 protein tyrosine kinases (PTKs) (Robinson et al., 2000), which can be divided into two major subtypes: receptor and non-receptor PTKs. Many PTKs are key enzymes in various critical signal transduction pathways and have important functions in the regulation of cellular processes such as cell growth, migration and differentiation. Overexpressed, mutated, or activated PTKs causes aberrant signaling and have been implicated in the pathogenesis of numerous diseases such as cancer, inflammatory disorders and diabetes (Hunter, 1997). Indeed, historically, PTKs constitute the prototypical class of oncogenes which have been found to be involved in most forms of human cancers. Therefore, PTKs are attractive drug discovery targets for cancer therapeutics. The recent clinical demonstration of therapeutic efficacy for several PTK inhibitors, e.g. Herceptin® which targets the HER-2/neu receptor, Tarceva® and Iressa® which target the EGF receptor, and STI-571 which targets BCR-ABL and KIT, provide important proof-of-concept for the validity of targeting PTKs for the treatment of cancer. Currently, a large and growing number of PTK targeting agents are under clinical evaluation.

The compound of Formula I (BMS-354825) is a potent inhibitor of several selected and related oncogenic PTKs: viz. BCR-ABL, c-SRC, c-KIT, PDGF receptor and EPH receptor. Each of these protein kinases has been strongly linked to multiple forms of human malignancies.

BCR-ABL, a fusion gene created as a consequence of a reciprocal translocation mutation in the long arms of Chromosome 9 and 12, encodes the BCR-ABL protein, a constitutively active cytoplasmic tyrosine kinase present in >90% of all patients with chronic myelogenous leukemia (CML) and in 15-30% of adult patients with acute lymphoblastic leukemia (ALL). Numerous studies have demonstrated that the activity of BCR-ABL is required for the cancer causing ability of this chimeric protein. With the recent clinical success and FDA approval of imatinib STI-571, the inhibition of BCR-ABL has been proven to be effective in the treatment of CML and has dramatically changed the treatment options for this disease. Currently CML patients can be broadly categorized into three subgroups: [1] patients in early (chronic) phase who are responsive to imatinib (the compound of Formula II), [2] patients in chronic phase who are imatinib-intolerant or resistant (innate or acquired), [3] patients in accelerated and blast crisis phase. For each of these populations there remain significant unmet medical needs.

Several groups have pointed out the appearance of imatinib-resistance in a significant proportion of CML patients, especially but not exclusively in advanced phases. Currently, in approximately 30% of patients with imatinib resistance, a mutation in the ABL-kinase domain of the BCR-ABL fusion gene is demonstrated. Even in responding ‘imatinib sensitive’ patients in complete cytogenetic remission (CCR), there remains still evidence of residual BCR-ABL+ leukemic progenitor cells in most patients and residual disease is rarely eliminated (Müller, M. C., Gattermann, N., Lahaye, T., Deininger, M. W. N., Berndt, A., Fruehauf, S., Neubauer, A., Fischer, T., Hossfeld, D. K., Schneller, F., Krause, S. W., Nerl, C., Sayer, H. G., Ottmann, O. G., Wailer, C., Aulitzky, W., Coutre, P. I., Freund, M., Merx, K., Paschka, P., König, H., Kreil, S., Berger, U., Gschaidmeier, H., Hehlmann, R. & Hochhaus, A. (2003). Dynamics of BCR-ABL mRNA expression in first-line therapy of chronic myelogenous leukemia patients with imatinib or interferon/ara-C. Leukemia, 17, 2392-2400).

Moreover, patients with advanced diseases (blast crisis) were much less sensitive to imatinib and responses when occurred were transient lasting less than 6 months (Druker et al., 2001). Clinical refractoriness to imatinib have been associated with the development of multiple mechanisms of drug resistance, including BCR-ABL gene mutation/overexpression (Shah et al., 2002) and activation of selected members of the SRC kinase family (Donato et al., 2003). Therefore, an urgent medical need clearly exists for more effective therapeutic option for CML, particularly for advanced diseases.

### SUMMARY OF THE INVENTION

The present invention provides a method for the treatment of cancer and/or leukemia, which comprises administering to a mammalian specie in need thereof a

therapeutically effective amount of: (1) at least one BCR-ABL inhibitor and (2) a compound of Formula I wherein the compound of Formula (I) is

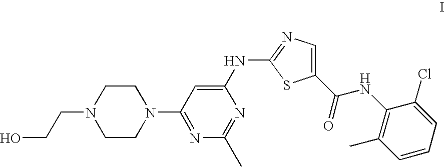

or pharmaceutically acceptable salt or crystalline form thereof.

A compound of Formula I is represented by ‘N-(2-Chloro-6-methylphenyl)-2-[[6-[4-(2-hydroxyethyl)-1-piperazinyl]-2-methyl-4-pyrimidinyl]amino]-5-thiazolecarboxamide and/or pharmaceutically acceptable salts or crystalline forms thereof.

The BCR-ABL inhibitor is represented by N-[5-[4-(4-methyl-piperazino-methyl)-benzoylamido]-2-methylphenyl}-4-(3-pyridyl)-2-pyrimidine-amine, the compound of Formula (II), also known as 4-(4-methyl-piperazin-1-ylmethyl)-N-[4-methyl-3-(4-pyridin-3-yl-pyrimidin-2-ylamino)-phenyl]-benzamide, STI571, imatinib, or under the marketed name Gleevec® (imatinib mesylate).

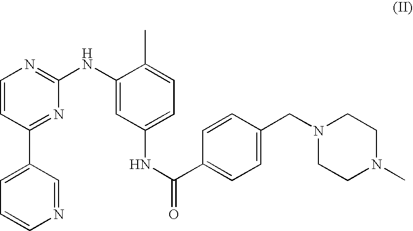

The present invention further provides a pharmaceutical composition for the treatment of cancer and/or leukemia which comprises a compound of Formula I, and a compound of Formula II, and a pharmaceutically acceptable carrier.

The present invention further provides a combination for the treatment of cancer and/or leukemia which comprises a therapeutically effective amount of a compound of Formula I or a pharmaceutically acceptable salt or crystalline form thereof, and a therapeutically effective amount of a compound of Formula II, or a pharmaceutically acceptable salt thereof.

In another embodiment of the invention the compound of Formula (II) is administered simultaneous with or before or after the administration of a compound of Formulas I.

### BRIEF DESCRIPTION OF THE DRAWINGS

The invention is illustrated by reference to the accompanying drawings described below.

FIG. 1 shows a simulated (bottom) (calculated from atomic coordinates generated at room temperature) and experimental (top) pXRD patterns for crystalline monohydrate of the compound of formula (I).

FIG. 2 shows a DSC and TGA of the of the monohydrate crystalline form of the compound of Formula (I).

FIG. 3 shows a simulated (bottom) (from atomic parameters refined at room temperature) and experimental (top) pXRD patterns for crystalline butanol solvate of the compound of formula (I).

FIG. 4 shows a simulated (bottom) (from atomic parameters refined at −40° C.) and experimental (top) pXRD patterns for crystalline ethanol solvate of the compound of formula (I).

FIG. 5 shows a simulated (bottom) (from atomic parameters refined at room temperature) and experimental (top) pXRD patterns for crystalline neat form (N−6) of the compound of formula (I).

FIG. 6 shows a simulated (bottom) (from atomic parameters refined at room temperature) and experimental (top) pXRD patterns for crystalline neat form (T1H1-7) of the compound of formula (I).

FIG. 7 shows a simulated (bottom) (from atomic parameters refined at room temperature) and experimental (top) pXRD patterns for ethanolate form (T1E2-1) of the compound of formula (I).

### DETAILED DESCRIPTION OF THE INVENTION

In accordance with the present invention, method for the treatment of cancer and/or leukemia, which comprises administering to a mammalian specie in need thereof a therapeutically effective amount of (1) a compound of Formula (I) or a pharmaceutically acceptable salt or crystalline form thereof,

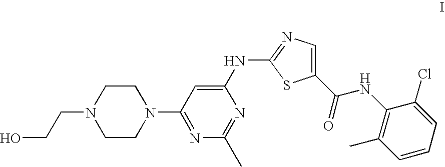

and 2) 4-(4-methyl-piperazin-1-ylmethyl)-N-[4-methyl-3-(4-pyridin-3-yl-pyrimidin-2-ylamino)-phenyl]-benzamide, (the compound of formula (II)) or a pharmaceutically acceptable salt thereof.

In another embodiment, the present invention is directed to a method of treating cancer and/or leukemia, wherein the cancer and/or leukemia is selected from chronic myelogenous leukemia (CML), acute lymphoblastic leukemia (ALL), and gastrointestinal stromal tumor (GIST), and acute myelogenous leukemia (AML).

In another embodiment, the present invention is directed to a method of treating cancer and/or leukemia, wherein 4-(4-methyl-piperazin-1-ylmethyl)-N-[4-methyl-3-(4-pyridin-3-yl-pyrimidin-2-ylamino)-phenyl]-benzamide, (the compound of formula (II)) is the mesylate salt.

In another embodiment, the present invention is directed to a method of treating cancer and/or leukemia, for the treatment of refractory cancers. An example of a refractory cancer is a cancer that is or has become resistant to other therapeutics, or is not effectively treated by the other therapeutic because of intolerance to the other therapeutic.

In another embodiment, the present invention is directed to a pharmaceutical composition which comprises a therapeutically effective amount of (1) a compound of Formula (I) or a pharmaceutically acceptable salt or crystalline form thereof,

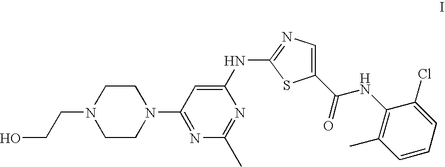

and 2) 4-(4-methyl-piperazin-1-ylmethyl)-N-[4-methyl-3-(4-pyridin-3-yl-pyrimidin-2-ylamino)-phenyl]-benzamide, (the compound of formula (II)) or a pharmaceutically acceptable salt thereof, and a pharmaceutically acceptable carrier.

In another embodiment, the present invention is directed to a pharmaceutical composition, wherein 4-(4-methyl-piperazin-1-ylmethyl)-N-[4-methyl-3-(4-pyridin-3-yl-pyrimidin-2-ylamino)-phenyl]-benzamide, (the compound of formula (II)) is the mesylate salt.

In another embodiment, the present invention is directed to a combination which comprises a therapeutically effective amount of (1) a compound of Formula (I) or a pharmaceutically acceptable salt or crystalline form thereof,

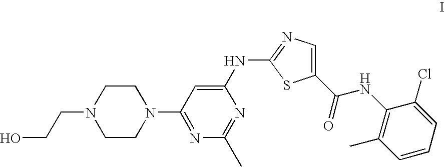

and 2) 4-(4-methyl-piperazin-1-ylmethyl)-N-[4-methyl-3-(4-pyridin-3-yl-pyrimidin-2-ylamino)-phenyl]-benzamide, (the compound of formula (II)) or a pharmaceutically acceptable salt thereof.

In another embodiment, the present invention is directed to the use of (l) a compound of Formula (I) or a pharmaceutically acceptable salt or crystalline form thereof,

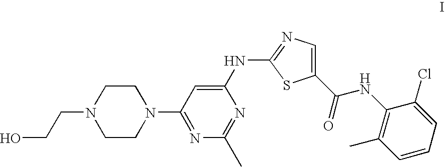

and 2) 4-(4-methyl-piperazin-1-ylmethyl)-N-[4-methyl-3-(4-pyridin-3-yl-pyrimidin-2-ylamino)-phenyl]-benzamide, (the compound of formula (II)) or a pharmaceutically acceptable salt thereof, in the manufacture of a medicament for the treatment of cancer and/or leukemia.

In another embodiment, the present invention is directed to a product comprising (1) a compound of Formula (I) or a pharmaceutically acceptable salt or crystalline form thereof,

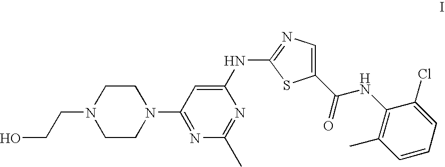

and 2) 4-(4-methyl-piperazin-1-ylmethyl)-N-[4-methyl-3-(4-pyridin-3-yl-pyrimidin-2-ylamino)-phenyl]-benzamide, (the compound of formula (II)) or a pharmaceutically acceptable salt thereof, as a combined preparation for simultaneous, separate or sequential use in therapy.

In another embodiment, the present invention is directed to the use of a compound of Formula (I) or a pharmaceutically acceptable salt or crystalline form thereof,

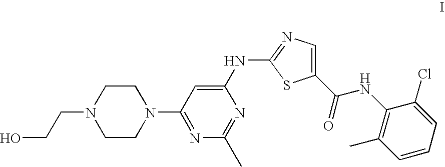

in the manufacture of a medicament for the treatment of cancer and/or leukemia, wherein the patient is also receiving treatment with 4-(4-methyl-piperazin-1-ylmethyl)-N-[4-methyl-3-(4-pyridin-3-yl-pyrimidin-2-ylamino)-phenyl]-benzamide, (the compound of formula (II)) or a pharmaceutically acceptable salt thereof.

In another embodiment, the present invention is directed to the use of 4-(4-methyl-piperazin-1-ylmethyl)-N-[4-methyl-3-(4-pyridin-3-yl-pyrimidin-2-ylamino)-phenyl]-benzamide, (the compound of formula (II)) or a pharmaceutically acceptable salt thereof, in the manufacture of a medicament for the treatment of cancer and/or leukemia, wherein the patient is also receiving treatment with a compound of Formula (I) or a pharmaceutically acceptable salt or crystalline form thereof

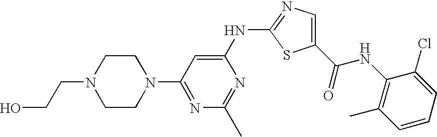

I. In another embodiment, the present invention is directed to the use of a compound of Formula (I) or a pharmaceutically acceptable salt or crystalline form thereof,

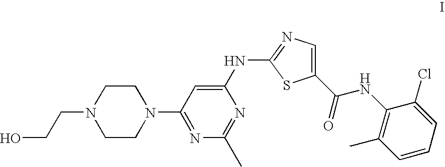

in the manufacture of a medicament for the treatment of cancer and/or leukemia, wherein the patient has been pre-treated with 4-(4-methyl-piperazin-1-ylmethyl)-N-[4-methyl-3-(4-pyridin-3-yl-pyrimidin-2-ylamino)-phenyl]-benzamide, (the compound of formula (II)) or a pharmaceutically acceptable salt thereof.

In another embodiment, the present invention is directed to a combination, wherein 4-(4-methyl-piperazin-1-ylmethyl)-N-[4-methyl-3-(4-pyridin-3-yl-pyrimidin-2-ylamino)-phenyl]-benzamide, (the compound of formula (II)) is the mesylate salt.

Thus, in an embodiment of the invention, the chemotherapeutic method of the invention comprises the administration of a Src Kinase Inhibitor of Formula I in combination with a BCR-ABL inhibitor.

A Src Kinase Inhibitors for use in the methods of the invention is a compound of Formula I wherein the compound of Formula I is

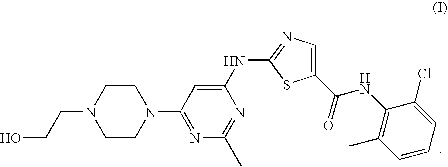

Compounds of the Formula I or Formula II may in some cases form salts which are also within the scope of this invention. Reference to a compound of the Formula I or Formula II herein is understood to include reference to salts thereof, unless otherwise indicated. The term “salt(s)”, as employed herein, denotes acidic and/or basic salts formed with inorganic and/or organic acids and bases. Zwitterions (internal or inner salts) are included within the term “salt(s)” as used herein (and may be formed, for example, where the R substituents comprise an acid moiety such as a carboxyl group). Also included herein are quaternary ammonium salts such as alkylammonium salts. Pharmaceutically acceptable (i.e., non-toxic, physiologically acceptable) salts useful, although other salts are useful, for example, in isolation or purification steps which may be employed during preparation. Salts of the compounds of the Formula I may be formed, for example, by reacting a compound I with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates, undecanoates, and the like.

Exemplary basic salts (formed, for example, where the R substituents comprise an acidic moiety such as a carboxyl group) include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines, N-methyl-D-glucamines, N-methyl-D-glucamides, t-butyl amines, and salts with amino acids such as arginine, lysine and the like. The basic nitrogen-containing groups may be quaternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

Prodrugs and solvates of the compounds of the invention are also contemplated herein. The term “prodrug”, as employed herein, denotes a compound which, upon administration to a subject, undergoes chemical conversion by metabolic or chemical processes to yield a compound of the Formula I, or a salt and/or solvate thereof. Solvates of the compounds of Formula I may be hydrates.

The combination of the present invention is intended to include the crystalline forms, such as hydrate, solvates and polymorphic forms of the compound of formula I. Therefore methods, pharmaceutical compositions, and combinations of the present invention are intended to include the crystalline forms of the compound of formula I as described below.

“Therapeutically effective amount” is intended to include an amount of a compound of the present invention alone or an amount of the combination of compounds claimed or an amount of a compound of the present invention in combination with other active ingredients effective to treat the diseases described herein.

A “synergistically, therapeutically effective amount” is a therapeutically effect amount which is provided by a synergistic combination.

The combination of the present invention may provide a synergistic effect useful for the treatment of leukemia and susceptible solid tumors. In another embodiment of this invention, a method is provided for the synergistic treatment of cancers including leukemia and solid tumors. Advantageously, the synergistic method of this invention reduces the development of tumors, reduces tumor burden, or produces tumor regression in a mammalian host.

The combinations of the compounds of the present invention are useful for the treatment of cancers such as chronic myelogenous leukemia (CML), acute lymphoblastic leukemia (ALL), gastrointestinal stromal tumor (GIST), acute myelogenous leukemia (AML), and others known to be associated with protein tyrosine kinases such as, for example, SPC, BCR-ABL and c-KIT. The combination of the compounds of the present invention are also useful in the treatment of cancers that are sensitive to and resistant to chemotherapeutic agents that target BCR-ABL and c-KIT, such as, for example, Gleevec® (STI-571) and AMN-107.

Methods for the safe and effective administration of most of the compounds of Formula I and II are known to those skilled in the art.

The methods of treating cancer and/or leukemia comprising the combination of the compounds of Formula (I) and Formula (II) of the present invention are useful for the treatment of patients wherein there remains evidence of residual BCR-ABL+ leukemic progenitor cells and residual disease following treatment by the compound of Formula (II) alone. Additionally, the combination of the compounds of Formula (I) and Formula (II) of the present invention are useful for the treatment of patients wherein there remains evidence of residual BCR-ABL+ leukemic progenitor cells and residual disease exhibits resistance to treatment by the compound of Formula (II) (by way of mutations which are not treated by the compound of Formula (II). Furthermore, the combination of the compounds of Formula (I) and Formula (II) are useful for the treatment of leukemia wherein the patient is resistant to treatment by the compound of Formula (II) alone.

Methods for the safe and effective administration of the compound of Formula (II) are known to those skilled in the art. For example, the administration of imatinib mesylate is described in the “Physicians' Desk Reference” (PDR), the disclosure of which is incorporated herein by reference thereto.

The compound of Formula I for use in the methods of the present invention is: ‘N-(2-Chloro-6-methylphenyl)-2-[[6-[4-(2-hydroxyethyl)-1-piperazinyl]-2-methyl-4-pyrimidinyl]amino]-5-thiazolecarboxamide; and pharmaceutically acceptable salts, solvates, hydrates and crystalline forms thereof.

The compounds of Formula I may be prepared by the procedures described in PCT publication, WO 00/62778 published Oct. 26, 2000, which is hereby incorporated by reference. The compound of formula I may be administered as described therein or as described in WO2004/085388, which is hereby incorporated by reference. The preparation of crystalline forms of the compound of formula I are described below and are described in U.S. application Ser. No. 11/015,208, filed Feb. 4, 2005, which is hereby incorporated by reference.

The preparation of the BCR-ABL inhibitor, 4-(4-methyl-piperazin-1-ylmethyl)-N-[4-methyl-3-(4-pyridin-3-yl-pyrimidin-2-ylamino)-phenyl]-benzamide, (the compound of formula (II)), is described in WO9903854 and may be administered as described therein. It may also be administered as marketed under the trademark Glivec™ or Gleevec®.

The present invention also encompasses a pharmaceutical composition useful in the treatment of cancer and/or leukemia, comprising the administration of a therapeutically effective amount of the combinations of this invention, with or without pharmaceutically acceptable carriers or diluents. The pharmaceutical compositions of this invention comprise a Formula I compound, an the compound 4-(4-methyl-piperazin-1-ylmethyl)-N-[4-methyl-3-(4-pyridin-3-yl-pyrimidin-2-ylamino)-phenyl]-benzamide, (the compound of formula (II)), and a pharmaceutically acceptable carrier. The compositions of the present invention may further comprise one or more pharmaceutically acceptable additional ingredients) such as alum, stabilizers, antimicrobial agents, buffers, coloring agents, flavoring agents, adjuvants, and the like. The compositions of the present invention may be administered orally or parenterally including the intravenous, intramuscular, intraperitoneal, subcutaneous, rectal and topical routes of administration.

For oral use, the compositions of this invention may be administered, for example, in the form of tablets or capsules, powders, dispersible granules, or cachets, or as aqueous solutions or suspensions. In the case of tablets for oral use, carriers which are commonly used include lactose, corn starch, magnesium carbonate, talc, and sugar, and lubricating agents such as magnesium stearate are commonly added. For oral administration in capsule form, useful carriers include lactose, corn starch, magnesium carbonate, talc, and sugar. When aqueous suspensions are used for oral administration, emulsifying and/or suspending agents are commonly added.

In addition, sweetening and/or flavoring agents may be added to the oral compositions. For intramuscular, intraperitoneal, subcutaneous and intravenous use, sterile solutions of the active ingredient(s) are usually employed, and the pH of the solutions should be suitably adjusted and buffered. For intravenous use, the total concentration of the solute(s) should be controlled in order to render the preparation isotonic.

For preparing suppositories according to the invention, a low melting wax such as a mixture of fatty acid glycerides or cocoa butter is first melted, and the active ingredient is dispersed homogeneously in the wax, for example by stirring. The molten homogeneous mixture is then poured into conveniently sized molds and allowed to cool and thereby solidify.

Liquid preparations include solutions, suspensions and emulsions. Such preparations are exemplified by water or water/propylene glycol solutions for parenteral injection. Liquid preparations may also include solutions for intranasal administration.

Aerosol preparations suitable for inhalation may include solutions and solids in powder form, which may be in combination with a pharmaceutically acceptable carrier, such as an inert compressed gas.

Also included are solid preparations which are intended for conversion, shortly before use, to liquid preparations for either oral or parenteral administration. Such liquid forms include solutions, suspensions and emulsions.

The composition described herein may also be delivered transdermally. The transdermal compositions can take the form of creams, lotions, aerosols and/or emulsions and can be included in a transdermal patch of the matrix or reservoir type as are conventional in the art for this purpose.

The combinations of the present invention may also be used in conjunction with other well known therapies that are selected for their particular usefulness against the condition that is being treated.

The effective amount of the compounds of the combination of the present invention may be determined by one of ordinary skill in the art, and includes exemplary dosage amounts for an adult human of from about 0.1 to 100 mg/kg of body weight of active compound per day, preferably at a dose from 1-50 mg/kg of body weight which may be administered in a single dose or in the form of individual divided doses, such as from 1 to 4 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Subjects for treatment include animals, most preferably mammalian species such as humans, and domestic animals such as dogs, cats and the like, subject to protein tyrosine kinase-associated disorders.

When administered intravenously, the compounds of the combination of the present invention, are preferably administered using the formulations of the invention.

As discussed above, compounds of the combination of the present invention, can be administered orally, intravenously, or both. In particular, the methods of the invention encompass dosing protocols such as once a day for 2 to 10 days, every 3 to 9 days, every 4 to 8 days and every 5 days. In one embodiment there is a period of 3 days to 5 weeks, 4 days to 4 weeks, 5 days to 3 weeks, and 1 week to 2 weeks, in between cycles where there is no treatment. In another embodiment the compounds of the combination of the present invention can be administered orally, intravenously, or both, once a day for 3 days, with a period of 3 week to 3 weeks in between cycles where there is no treatment. In yet another embodiment the compounds of the combination of the present invention can be administered orally, intravenously, or both, once a day for 5 days, with a period of 1 week to 3 weeks in between cycles where there is no treatment.

In one embodiment the treatment cycle for administration of the compounds of the combination of the present invention, is once daily for 5 consecutive days and the period between treatment cycles is from 2 to 10 days, or one week. In one embodiment, a combination of the compound of the present invention, is administered once daily for 5 consecutive days, followed by 2 days when there is no treatment.

The compounds of the combination of the present invention can also be administered orally, intravenously, or both once every 1 to 10 weeks, every 2 to 8 weeks, every 3 to 6 weeks, and every 3 weeks.

The combination of the compounds of Formula I and Formula II may be formulated as a fixed dose. Alternatively, the active ingredients may be administered separately. In another embodiment of the present invention, the compound of formula II is administered following or simultaneously with administration of the Formula I compound.

In another embodiment of the invention, the compound of formula I may be administered in a dose of 15-200 mg twice a day, or 30-100 mg twice a day. In one embodiment, the compound of formula I may be administered at 70 mg twice a day. In another embodiment, the compound of formula I may be administered in a dose of 50-300 mg once a day, or 100-200 mg once a day. Alternatively, the compound of formula I may be administered in a dose of 75-150 mg twice a day or 140-250 mg once a day. Alternatively, the compound of formula I may be administered at 50, 60, 70, 80, 90, 100, 110, 120, 130 or 140 mg twice a day, or doses in between. Alternatively, the compound of formula I may be administered at 100, 120, 140, 160, 180, 200, 220 or 240 mg once a day, or doses in between. The compound of formula I may be administered either continuously or on an alternating schedule, such as 5 days on, 2 days off, or some other schedule as described above.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

When employing the methods or compositions of the present invention, other agents used in the modulation of tumor growth or metastasis in a clinical setting, such as antiemetics, can also be administered as desired.

The present invention encompasses a method for the treatment of cancer and/or leukemia wherein a compound of Formula I and a compound of Formula II compound are administered simultaneously or sequentially. Thus, while a pharmaceutical formulation comprising a compound of Formula II and a Formula I compound may be advantageous for administering the combination for one particular treatment, prior administration of the compound of Formula II may be advantageous in another treatment. It is also understood that the instant combination the compound of Formula II and Formula I compound may be used in conjunction with other methods of treating cancer (such as cancerous tumors) including, but not limited to, radiation therapy and surgery. It is further understood that a cytostatic or quiescent agent, if any, may be administered sequentially or simultaneously with any or all of the other therapies. It is further understood that the routes of administration may vary between the compounds of Formula I and the compound of Formula II.

The combinations of the instant invention may also be co-administered with other well known therapeutic agents that are selected for their particular usefulness against the condition that is being treated. Combinations of the instant invention may alternatively be used sequentially with known pharmaceutically acceptable agent(s) when a multiple combination formulation is inappropriate.

The chemotherapeutic agent(s) can be administered according to therapeutic protocols well known in the art. It will be apparent to those skilled in the art that the administration of the chemotherapeutic agent(s) can be varied depending on the disease being treated and the known effects of the chemotherapeutic agent(s). Also, in accordance with the knowledge of the skilled clinician, the therapeutic protocols (e.g., dosage amounts and times of administration) can be varied in view of the observed effects of the administered therapeutic agents (i.e., antineoplastic agent(s) or radiation) on the patient, and in view of the observed responses of the disease to the administered therapeutic agents.

In the methods of this invention, a compound of Formula I is administered simultaneously or sequentially with a compound of Formula II. Thus, it is not necessary that the compound of Formula II and the compound of Formula I, be administered simultaneously or essentially simultaneously. The advantage of a simultaneous or essentially simultaneous administration is well within the determination of the skilled clinician.

Also, in general, the compound of Formula I, and the compound of Formula II do not have to be administered in the same pharmaceutical composition, and may, because of different physical and chemical characteristics, have to be administered by different routes. For example, the compound of Formula I may be administered orally to generate and maintain good blood levels thereof, while the compound of Formula II may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of compound of Formula I and the compound of Formula II will depend upon the diagnosis of the attending physicians and their judgment of the condition of the patient and the appropriate treatment protocol.

If the compound of Formula I and the compound of Formula II are not administered simultaneously or essentially simultaneously, then the initial order of administration of the compound of Formula I, and the compound of Formula II, may be varied. Thus, for example, the compound of Formula I may be administered first followed by the administration of the compound of Formula II; or the compound of Formula II may be administered first followed by the administration of the compound of Formula I. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient.

Thus, in accordance with experience and knowledge, the practicing physician can modify each protocol for the administration of a component (therapeutic agent—i.e., compound of Formula I, compound of Formula II) of the treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

The above combination of the compounds of Formula (I) and Formula (II) are useful to effectively treat leukemias that previously had developed a resistance to such drugs. Additionally, the present inventors have developed methods for the treatment of cancer which permit the clinician to administer lowered dosages of anticancer agents with appropriate administration schedules thereby reducing unwanted side effects while maintaining efficacy.

The preparation of crystalline forms of the compound of Formula I are described below. The present invention is intended to include in the combinations as described above crystalline forms of the compound of formula I.

### Analytical Methods

### Solid State Nuclear Magnetic Resonance (SSNMR)

All solid-state C-13 NMR measurements were made with a Bruker DSX-400, 400 MHz NMR spectrometer. High resolution spectra were obtained using high-power proton decoupling and the TPPM pulse sequence and ramp amplitude cross-polarization (RAMP-CP) with magic-angle spinning (MAS) at approximately 12 kHz (A. E. Bennett et al, J. Chem. Phys., 1995, 103, 6951), (G. Metz, X. Wu and S. O. Smith, J. Magn. Reson. A., 1994, 110, 219-227). Approximately 70 mg of sample, packed into a canister-design zirconia rotor was used for each experiment. Chemical shifts (δ) were referenced to external adamantane with the high frequency resonance being set to 38.56 ppm (W. L. Earl and D. L. VanderHart, J. Magn. Reson., 1982, 48, 35-54).

### X-Ray Powder Diffraction

One of ordinary skill in the art will appreciate that an X-ray diffraction pattern may be obtained with a measurement error that is dependent upon the measurement conditions employed. In particular, it is generally known that intensities in a X-ray diffraction pattern may fluctuate depending upon measurement conditions employed.

It should be further understood that relative intensities may also vary depending upon experimental conditions and, accordingly, the exact order of intensity should not be taken into account. Additionally, a measurement error of diffraction angle for a conventional X-ray diffraction pattern is typically about 5% or less, and such degree of measurement error should be taken into account as pertaining to the aforementioned diffraction angles. Consequently, it is to be understood that the crystal forms of the instant invention are not limited to the crystal forms that provide X-ray diffraction patterns completely identical to the X-ray diffraction patterns depicted in the accompanying Figures disclosed herein. Any crystal forms that provide X ray diffraction patterns substantially identical to those disclosed in the accompanying Figures fall within the scope of the present invention. The ability to ascertain substantial identities of X-ray diffraction patterns is within the purview of one of ordinary skill in the art.

X-Ray powder diffraction data for the crystalline forms of Compound (I) were obtained using a Bruker GADDS (BRUKER AXS, Inc., 5465 East Cheryl Parkway Madison, Wis. 53711 USA) (General Area Detector Diffraction System) manual chi platform goniometer. Powder samples were placed in thin walled glass capillaries of 1 mm or less in diameter; the capillary was rotated during data collection. The sample-detector distance was 17 cm. The radiation was Cu Kα (45 kV 111 mA, λ=1.5418 Å). Data were collected for 3<2θ<35° with a sample exposure time of at least 300 seconds.

### Single Crystal X-Ray

All single crystal data were collected on a Bruker-Nonius (BRUKER AXS, Inc., 5465 East Cheryl Parkway Madison, Wis. 53711 USA) Kappa CCD 2000 system using Cu Kα radiation (λ=1.5418 Å) and were corrected only for the Lorentz-polarization factors. Indexing and processing of the measured intensity data were carried out with the HKL2000 software package (Otwinowski, Z. & Minor, W. (1997) in Macromolecular Crystallography, eds. Carter, W. C. Jr & Sweet, R. M. (Academic, N.Y.), Vol. 276, pp. 307-326) in the Collect program suite (Data collection and processing user interface: Collect: Data collection software, R. Hooft, Nonius B. V., 1998).

The structures were solved by direct methods and refined on the basis of observed reflections using either the SDP (SDP, Structure Determination Package, Enraf-Nonius, Bohemia N.Y. 11716 Scattering factors, including f′ and f″, in the SDP software were taken from the “International Tables for Crystallography”, Kynoch Press, Birmingham, England, 1974; Vol IV, Tables 2.2A and 2.3.1) software package with minor local modifications or the crystallographic package, MAXUS (maxus solution and refinement software suite: S. Mackay, C. J. Gilmore, C. Edwards, M. Tremayne, N. Stewart, K. Shankland. maXus: a computer program for the solution and refinement of crystal structures from diffraction data).

The derived atomic parameters (coordinates and temperature factors) were refined through full matrix least-squares. The function minimized in the refinements was Σ_{W}(|F_{O}|−|F_{C}|)². R is defined as Σ∥F_{O}|−|F_{C}∥/Σ|F_{O}| while R_{W}=[Σ_{W}(|F_{O}|−|F_{C}|)²/Σ_{W}|F_{O}|²]^{1/2} where w is an appropriate weighting function based on errors in the observed intensities. Difference maps were examined at all stages of refinement. Hydrogens were introduced in idealized positions with isotropic temperature factors, but no hydrogen parameters were varied.

The derived atomic parameters (coordinates and temperature factors) were refined through full matrix least-squares. The function minimized in the refinements was Σ_{W}(|F_{O}|−|F_{C}|)². R is defined as Σ∥F_{O}|−|F_{C}∥/Σ|F_{O}| while R=[Σ_{W}(|F_{O}|−|F_{C}|)₂/Σ_{W}|F_{O}|²]^{1/2} where w is an appropriate weighting function based on errors in the observed intensities. Difference maps were examined at all stages of refinement. Hydrogens were introduced in idealized positions with isotropic temperature factors, but no hydrogen parameters were varied

### Differential Scanning Calorimetry

The DSC instrument used to test the crystalline forms was a TA Instruments® model Q1000. The DSC cell/sample chamber was purged with 100 ml/min of ultra-high purity nitrogen gas. The instrument was calibrated with high purity indium. The accuracy of the measured sample temperature with this method is within about +/−1° C., and the heat of fusion can be measured within a relative error of about +/−5%. The sample was placed into an open aluminum DSC pan and measured against an empty reference pan. At least 2 mg of sample powder was placed into the bottom of the pan and lightly tapped down to ensure good contact with the pan. The weight of the sample was measured accurately and recorded to a hundredth of a milligram. The instrument was programmed to heat at 10° C. per minute in the temperature range between 25 and 350° C.

The heat flow, which was normalized by a sample weight, was plotted versus the measured sample temperature. The data were reported in units of watts/gram (“W/g”). The plot was made with the endothermic peaks pointing down. The endothermic melt peak was evaluated for extrapolated onset temperature, peak temperature, and heat of fusion in this analysis.

### Thermogravimetric Analysis (TGA)

The TGA instrument used to test the crystalline forms was a TA Instruments® model Q500. Samples of at least 10 milligrams were analyzed at a heating rate of 10° C. per minute in the temperature range between 25° C. and about 350° C.

### EXAMPLE 1

### Preparation of

### crystalline monohydrate of N-(2-chloro-6-methylphenyl)-2-(6-(4-(3-hydroxyethyl)piperazin-1-yl)-2-methylpyrimidin-4-ylamino) thiazole-5-carboxamide (I)

An example of the crystallization procedure to obtain the crystalline monohydrate form is shown here:

Charge 48 g of the compound of formula (I).
Charge approximately 1056 mL (22 mL/g) of ethyl alcohol, or other suitable alcohol.

Charge approximately 144 mL of water.

Dissolve the suspension by heating to approximately 75° C.

Optional: Polish filter by transfer the compound of formula (I) solution at 75° C. through the preheated filter and into the receiver.

Rinse the dissolution reactor and transfer lines with a mixture of 43 mL of ethanol and 5 mL of water.

Heat the contents in the receiver to 75-80° C. and maintain 75-80° C. to achieve complete dissolution.

Charge approximately 384 mL of water at a rate such that the batch temperature is maintained between 75-80° C.

Cool to 75° C. and, optionally, charge monohydrate seed crystals. Seed crystals are not essential to obtaining monohydrate, but provide better control of the crystallization.

Cool to 70° C. and maintain 70° C. for ca. 1 h.

Cool from 70 to 5 C over 2 h, and maintain the temperature between 0 at 5° C. for at least 2 h.

Filter the crystal slurry.

Wash the filter cake with a mixture of 96 mL of ethanol and 96 mL of water.

Dry the material at ≦50° C. under reduced pressure until the water content is 3.4 to 4.1% by KF to afford 41 g (85 M %).

Alternately, the monohydrate can be obtained by:
1) An aqueous solution of the acetate salt of compound I was seeded with monohydrate and heated at 80° C. to give bulk monohydrate.
2) An aqueous solution of the acetate salt of compound I was seeded with monohydrate. On standing several days at room temperature, bulk monohydrate had formed.
3) An aqueous suspension of compound I was seeded with monohydrate and heated at 70° C. for 4 hours to give bulk monohydrate. In the absence of seeding, an aqueous slurry of compound I was unchanged after 82 days at room temperature.
4) A solution of compound I in a solvent such as NMP or DMA was treated with water until the solution became cloudy and was held at 75-85° C. for several hours. Monohydrate was isolated after cooling and filtering.
5) A solution of compound I in ethanol, butanol, and water was heated. Seeds of monohydrate were added to the hot solution and then cooled. Monohydrate was isolated upon cooling and filtration.

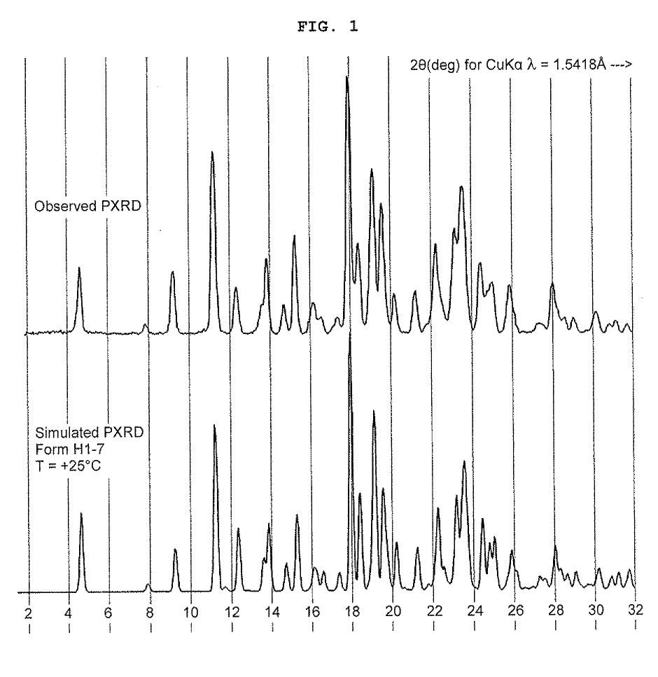

One of ordinary skill in the art will appreciate that the monohydrate of the compound of formula (I) may be represented by the XRPD as shown in FIG. 1 or by a representative sampling of peaks as shown in Table 1.

Representative peaks taken from the XRPD of the monohydrate of the compound of formula (I) are shown in Table 1.

TABLE 1

| 2-Theta | d(Å) | Height |
|---|---|---|
| 17.994 | 4.9257 | 915 |
| 18.440 | 4.8075 | 338 |
| 19.153 | 4.6301 | 644 |
| 19.599 | 4.5258 | 361 |
| 21.252 | 4.1774 | 148 |
| 24.462 | 3.6359 | 250 |
| 25.901 | 3.4371 | 133 |
| 28.052 | 3.1782 | 153 |

The XRPD is also characterized by the following list comprising 2θ values selected from the group consisting of: 4.6±0.2, 11.2±0.2, 13.8±0.2, 15.2±0.2, 17.9±0.2, 19.1±0.2, 19.6±0.2, 23.2±0.2, 23.6±0.2. The XRPD is also characterized by the list of 2θ values selected from the group consisting of: 18.0±0.2, 18.4±0.2, 19.2±0.2, 19.6±0.2, 21.2±0.2, 24.5±0.2, 25.9±0.2, and 28.0±0.2.

Single crystal x-ray data was obtained at room temperature (+25° C.). The molecular structure was confirmed as a monohydrate form of the compound of Formula (I).

The following unit cell parameters were obtained for the monohydrate of the compound of formula (I) from the x-ray analysis at 25° C.:
a(↑1)=13.8632(7); b(Å)=9.3307(3); c(Å)=38.390(2); V(Å³) 4965.9(4); Z′=1; Vm=621
Space group Pbca
Molecules/unit cell 8
Density (calculated) (g/cm³) 1.354
   Wherein Z′=number of drug molecules per asymmetric unit.

Vm=V(unit cell)/(Z drug molecules per cell).

Single crystal x-ray data was also obtained at 50° C. The monohydrate form of the compound of Formula (I) is characterized by unit cell parameters approximately equal to the following:

Cell dimensions:
a(Å)=13.862(1);
b(Å)=9.286(1);
c(Å)=38.143(2);
Volume=4910(1) Å³
Space group Pbca
Molecules/unit cell 8
Density (calculated) (g/cm³) 1.369
   wherein the compound is at a temperature of about −50° C.

The simulated XRPD was calculated from the refined atomic parameters at room temperature.

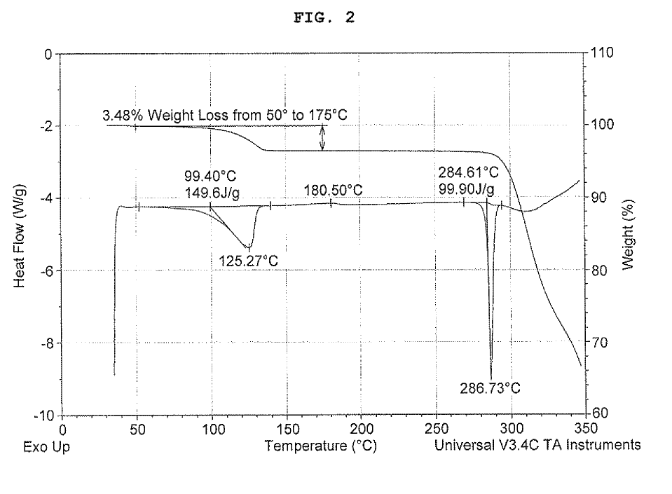

The monohydrate of the compound of formula (I) is represented by the DSC as shown in FIG. 2. The DSC is characterized by a broad peak between approximately 95° C. and 130° C. This peak is broad and variable and corresponds to the loss of one water of hydration as seen in the TGA graph. The DSC also has a characteristic peak at approximately 287° C. which corresponds to the melt of the dehydrated form of the compound of formula (I).

The TGA for the monohydrate of the compound of Formula (I) is shown in FIG. 2 along with the DSC. The TGA shows a 3.48% weight loss from 50° C. to 175° C. The weight loss corresponds to a loss of one water of hydration from the compound of Formula (I).

The monohydrate may also be prepared by crystallizing from alcoholic solvents, such as methanol, ethanol, propanol, i-propanol, butanol, pentanol, and water.

### EXAMPLE 2

### Preparation of

### crystalline n-butanol solvate of N-(2-chloro-6-methylphenyl)-2-(6-(4-(3-hydroxyethyl)piperazin-1-yl)-2-methylpyrimidin-4-ylamino) thiazole-5-carboxamide (I)

The crystalline butanol solvate of the compound of formula (I) is prepared by dissolving compound (I) in 1-butanol at reflux (116-118° C.) at a concentration of approximately 1 g/25 mL of solvent. Upon cooling, the butanol solvate crystallizes out of solution. Filter, wash with butanol, and dry.

The following unit cell parameters were obtained from the x-ray analysis for the crystalline butanol solvate, obtained at room temperature:

a(Å)=22.8102(6);

b(Å)=8.4691(3);

c(Å)=15.1436(5);

β=95.794(2);

V(Å³) 2910.5(2); Z′=1; Vm=728

Space group P2₁/a

Molecules/unit cell 4

Density (calculated) (g/cm³) 1.283

Wherein Z′=number of drug molecules per asymmetric unit.

Vm=V(unit cell)/(Z drug molecules per cell).

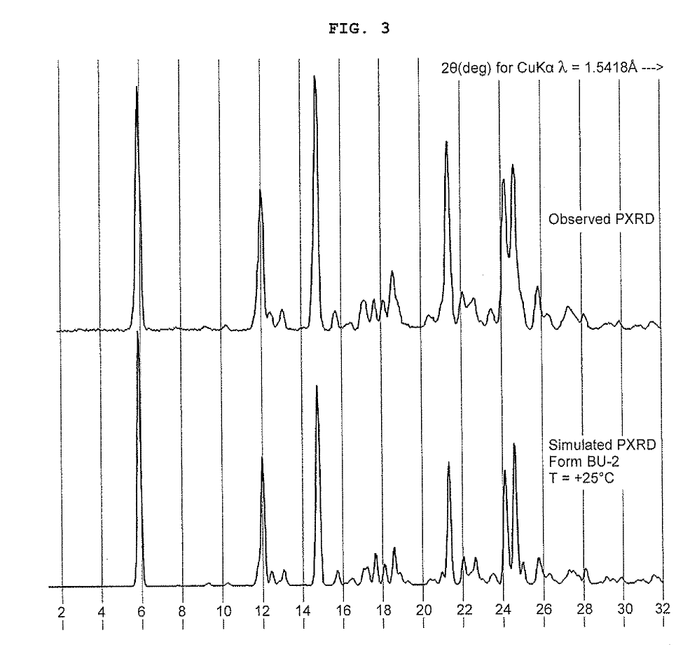

One of ordinary skill in the art will appreciate that the butanol solvate of the compound of formula (I) may be represented by the XRPD as shown in FIG. 3 or by a representative sampling of peaks. Representative peaks for the crystalline butanol solvate are 2□, values of: 5.9±0.2, 12.0±0.2, 13.0±0.2, 17.7±0.2, 24.1±0.2, and 24.6±0.2.

### EXAMPLE 3

### Preparation of

### crystalline ethanol solvate of N-(2-chloro-6-methylphenyl)-2-(6-(4-(3-hydroxyethyl)piperazin-1-yl)-2-methylpyrimidin-4-ylamino) thiazole-5-carboxamide (I)

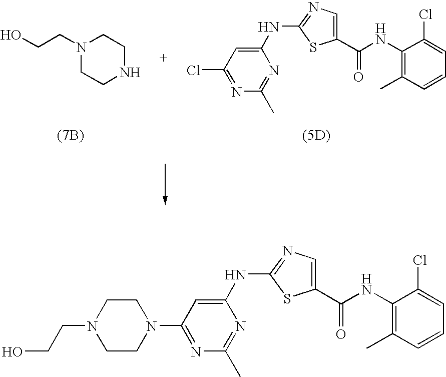

To a 100-mL round bottom flask was charged 4.00 g (10.1 mmol) of 5D (contained 2.3 Area % 5C) 6.60 g (50.7 mmol) of 7B, 80 mL of n-butanol and 2.61 g (20.2 mmol) of DIPEA. The resulting slurry was heated to 120° C. and maintained at 120° C. for 4.5 h whereby HPLC analysis showed 0.19 relative Area % of residual 5D to compound IV.

The homogeneous mixture was cooled to 20° C. and left stirring overnight. The resulting crystals were filtered. The wet cake was washed twice with 10-mL portions of n-butanol to afford a white crystalline product. HPLC analysis showed this material to contain 99.7 Area % compound IV and 0.3 Area % 5C.

The resulting wet cake was returned to the 100-mL reactor, and charged with 56 mL (12 mL/g) of 200 proof ethanol. At 80° C. an additional 25 mL of ethanol was added. To this mixture was added 10 mL of water resulting in rapid dissolution. Heat was removed and crystallization was observed at 75-77° C. The crystal slurry was further cooled to 20° C. and filtered. The wet cake was washed once with 10 mL of 1:1 ethanol:water and once with 10 mL of n-heptane. The wet cake contained 1.0% water by KF and 8.10% volatiles by LOD. The material was dried at 60° C./30 in Hg for 17 h to afford 3.55 g (70 M %) of material containing only 0.19% water by KF, 99.87 Area % by HPLC. The ¹H NMR spectrum, however revealed that the ethanol solvate had been formed.

The following unit cell parameters were obtained from the x-ray analysis for the crystalline ethanol solvate (di-ethanolate, E2-1), obtained at −40° C.:

a(Å)=22.076(1); b(Å)=8.9612(2); c(Å)=16.8764(3); β=114.783(1);

V(Å³) 3031.1(1); Z′=1; Vm=758

Space group P2₁/a

Molecules/unit cell 4

Density (calculated) (g/cm³) 1.271

Wherein Z′=number of drug molecules per asymmetric unit.

Vm=V(unit cell)/(Z drug molecules per cell).

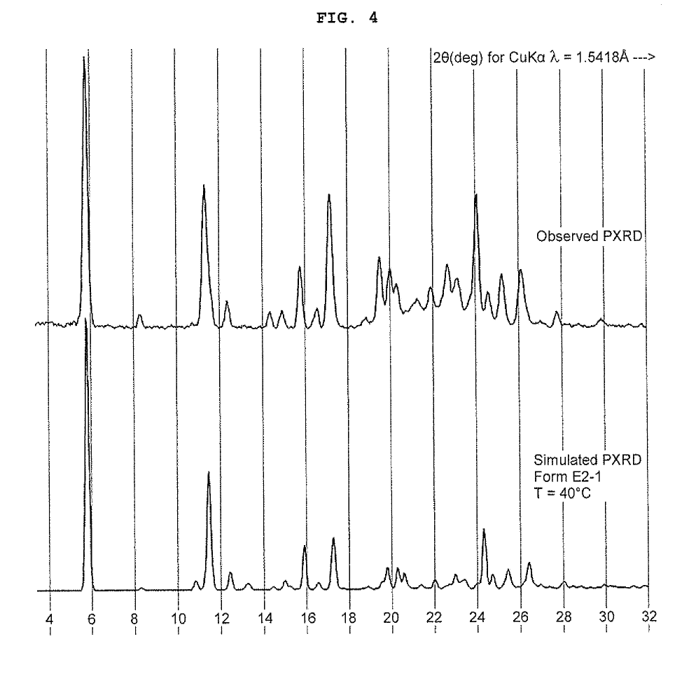

One of ordinary skill in the art will appreciate that the ethanol solvate (E2-1) of the compound of formula (I) may be represented by the XRPD as shown in FIG. 4 or by a representative sampling of peaks. Representative peaks for the crystalline ethanol solvate are 20, values of 5.8±0.2, 11.3±0.2, 15.8±0.2, 17.2±0.2, 19.5±0.2, 24.1±0.2, 25.3±0.2, and 26.2±0.2.

In addition, during the process to form the ethanolate (diethanolate) the formation of another ethanol solvate (½ ethanolate, T1E2-1) has been observed. To date this additional ethanol solvate is known strictly as a partial desolvation product of the original diethanolate form E2-1, and has only been observed on occasion during crystallization of E2-1

The following unit cell parameters were obtained from the x-ray analysis for the crystalline ½ ethanol solvate T1E2-1, obtained at −10° C.:
a(Å)=22.03(2); b(Å)=9.20(1); c(Å)=12.31(1); β=93.49(6)
V(Å³) 2491(4)); Z′=1; Vm=623;
Space group P2₁/a
Molecules/unit cell 4
Density (calculated) (g/cm³) 1.363
   Wherein Z′=number of drug molecules per asymmetric unit.

Vm=V(unit cell)/(Z drug molecules per cell).

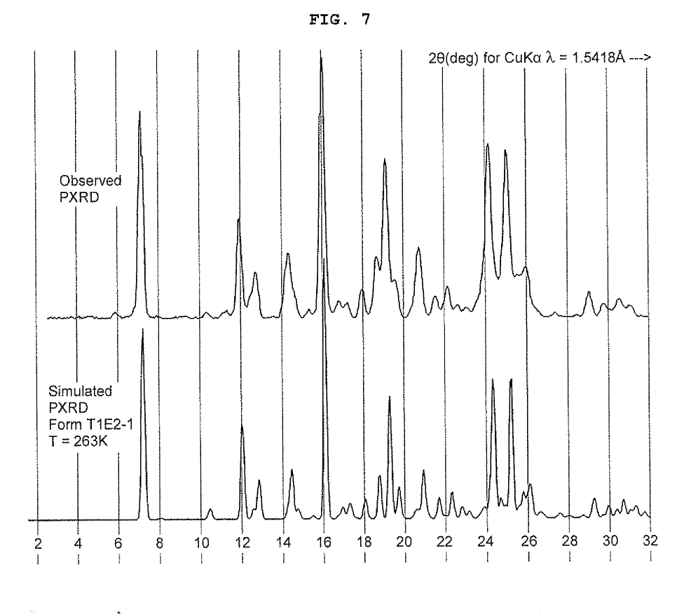

One of ordinary skill in the art will appreciate that the ethanol solvate (T1E2-1) of the compound of formula (I) may be represented by the XRPD as shown in FIG. 7 or by a representative sampling of peaks. Representative peaks for the crystalline ethanol solvate are 2θ values of: 7.20±0.2, 12.01±0.2, 12.81±0.2, 18.06±0.2, 19.30±0.2, and 25.24±0.2.

### EXAMPLE 4

### Preparation of

### crystalline N-(2-chloro-6-methylphenyl) 2-(6-(4-(3-hydroxyethyl)piperazin-1-yl)-2-methylpyrimidin-4-ylamino) thiazole-5-carboxamide (I) (Neat form N-6)

To a mixture of compound 5D (175.45 g, 0.445 mol) and hydroxyethylpiperazine (289.67 g, 2.225 mol) in NMP (1168 mL) was added DIPEA (155 mL, 0.89 mol). The suspension was heated at 110° C. (solution obtained) for 25 min., then cooled to about 90° C. The resulting hot solution was added dropwise into hot (80° C.) water (8010) mL, keeping the temperature at about 80° C. The resulting suspension was stirred 15 min at 80° C. then cooled slowly to room temperature. The solid was collected by vacuum filtration, washed with water (2×1600 mL) and dried in vacuo at 55-60° C. affording 192.45 g (88.7% yield) of N-(2-chloro-6-methylphenyl)-2-(6-(4-(3-hydroxyethyl)piperazin-1-yl)-2-methylpyrimidin-4-ylamino)thiazole-5-carboxamide. ¹H NMR (400 MHz, DMSO-d₆): δ 2.24 (s, 3H), 2.41 (s, 3H), 2.43 (t, 2H, J=6), 2.49 (t, 4H, J=6.3), 3.51 (m, 4H), 3.54 (q, 2H, J=6), 4.46 (t, 1H, J=5.3), 6.05 (s, 1H), 7.26 (t, 1H, J=7.6), 7.28 (dd, 1H, J=7.6, 1.7), 7.41 (dd, 1H, J=7.6, 1.7), 8.23 (s, 1H), 9.89 (S, 1H), 11.48. KF0.84; DSC: 285.25° C. (onset), 286.28° C. (max).

The following unit cell parameters were obtained from the x-ray analysis for the neat crystalline compound IV, obtained at 23° C.:

a(Å)=22.957(1); b(Å)=8.5830(5); c(Å) 13.803(3); β=112.039(6);

V(Å³) 2521.0(5); Z′=1; Vm=630

Space group P2₁/a

Molecules/unit cell 4

Density (calculated) (g/cm³) 1.286

Wherein Z′=number of drug molecules per asymmetric unit.

Vm=V(unit cell)/(Z drug molecules per cell).

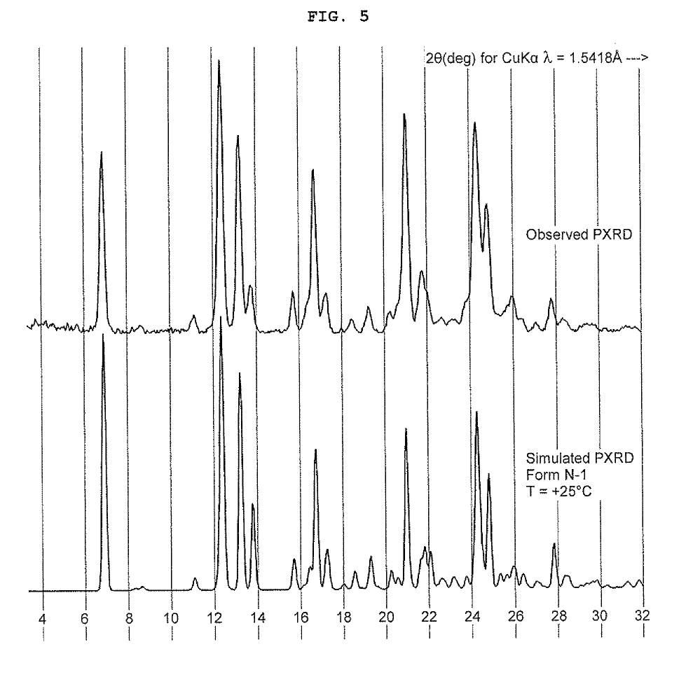

One of ordinary skill in the art will appreciate that the crystalline form of the compound of formula (I) may be represented by the XRPD as shown in FIG. 5 or by a representative sampling of peaks. Representative peaks for the crystalline neat form (N−6) are 2θ values of: 6.8±0.2, 11.1±0.2, 12.3±0.2, 13.2±0.2, 13.7±0.2, 16.7±0.2, 21.0±0.2, 24.3±0.2, and 24.8±0.2.

### EXAMPLE 5

### Preparation of

### crystalline N-(2-chloro-6-methylphenyl) 2-(6-(4-(3-hydroxyethyl)piperazin-1-yl)-2-methylpyrimidin-4-ylamino)thiazole-5-carboxamide (I) (neat form T1H1-7)

The title neat form may be prepared by heating the monohydrate form of the compound of formula (I) above the dehydration temperature.

The following unit cell parameters were obtained from the x-ray analysis for the neat crystalline (T1H1-7) compound IV, obtained at 25° C.:
a(Å)=13.4916; b(Å)=9.3992(2); c(Å)=38.817(1); V(Å³) 4922.4(3); Z′=1; Vm=615
Space group Pbca
Density (calculated) (g/cm³) 1.317
   Wherein Z′=number of drug molecules per asymmetric unit.

Vm=V(unit cell)/(Z drug molecules per cell).

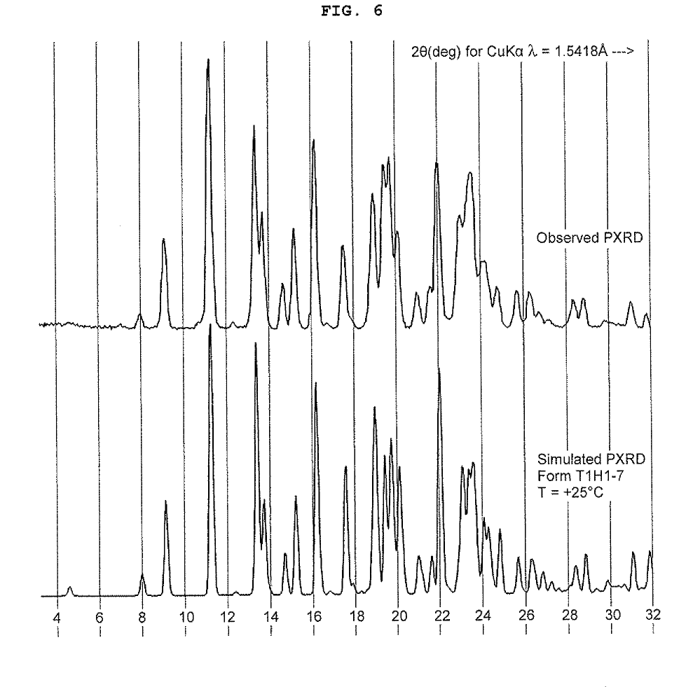

One of ordinary skill in the art will appreciate that the neat crystalline form (T1H1-7) of the compound of formula (I) may be represented by the XRPD as shown in FIG. 6 or by a representative sampling of peaks, Representative peaks for the crystalline neat form (T1H1-7)) are 2θ values of: 8.0±0.2, 9.7±0.2, 11.2±0.2, 13.3±0.2, 17.5±0.2, 18.9±0.2, 21.0±0.2, 22.0±0.2.

What is claimed is:

1. A method for the treatment of gastrointestinal stromal tumor (GIST) and/or leukemia, which comprises administering to a mammalian specie in need thereof a therapeutically effective amount of (1) a compound of formula (I) or a pharmaceutically acceptable salt, hydrate, solvate, or crystalline form thereof, 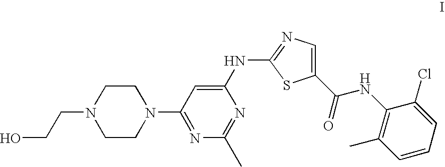
and 2) a compound of formula II or a pharmaceutically acceptable salt thereof

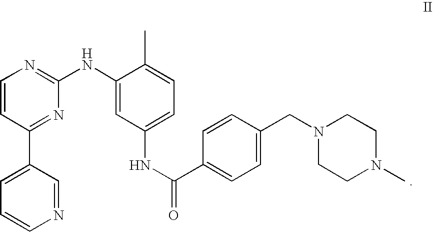

2. The method of claim 1 wherein the leukemia is selected from chronic myelogenous leukemia (CML), acute lymphoblastic leukemia (ALL), and acute myelogenous leukemia (AML).

3. The method of claim 1, wherein the compound of formula II is the mesylate salt.

4. The method according to claim 1, wherein the gastrointestinal and/or leukemia is a refractory gastrointestinal stromal tumor (GIST) and/or leukemia.

5. A pharmaceutical composition which comprises a therapeutically effective amount of (1) a compound of Formula (I) or a pharmaceutically acceptable salt, hydrate, solvate, or crystalline form thereof, 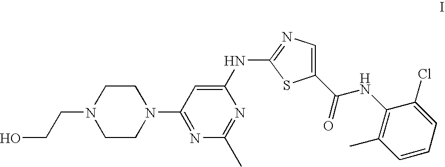
and 2) N-[5-[4-(4-methyl-piperazino-methyl)-benzoylamido]-2-methylphenyl}-4-(3-pyridyl)-2-pyrimidine-amine or a pharmaceutically acceptable salt thereof, and a pharmaceutically acceptable carrier.

6. The method of claim 5, wherein N-[5-[4-(4-methyl-piperazino-methyl)-benzoylamido]-2-methylphenyl}-4-(3-pyridyl)-2-pyrimidine-amine is the mesylate salt.

7. A combination which comprises a therapeutically effective amount of (1) a compound of Formula (I) or a pharmaceutically acceptable salt, hydrate, solvate, or crystalline form thereof, 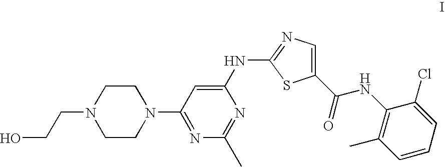
and 2) N-[5-[4-(4-methyl-piperazino-methyl)-benzoylamido]-2-methylphenyl}-4-(3-pyridyl)-2-pyrimidine-amine or a pharmaceutically acceptable salt thereof.

8. The combination of claim 7, wherein N-[5-[4-(4-methyl-piperazino-methyl)-benzoylamido]-2-methylphenyl}-4-(3-pyridyl)-2-pyrimidine-amine is the mesylate salt.

9. The method according to claim 1, wherein the leukemia is a refractory leukemia and the leukemia is selected from chronic myelogenous leukemia (CML), acute lymphoblastic leukemia (ALL), and acute myelogenous leukemia (AML).

10. The method according to claim 1, wherein the compound of formula I and the compound of formula II or a pharmaceutically acceptable salt of either are administered separately, simultaneously, or sequentially.
